(19) United States
(12) Patent Application Publication
(10) Pub. No.: US 2020/0124625 A1
(43) Pub. Date: Apr. 23, 2020

(54) SYSTEMS AND METHODS FOR DETECTING A TARGET ANALYTE IN A BREATH SAMPLE

(71) Applicant: SannTek Labs Inc., Kitchener (CA)

(72) Inventors: Thomas Dunlop, Milton (CA); Noah Debrincat, Georgetown (CA); Mustafa Yuksel, Waterloo (CA); Karolyn Mackowiak, Richmond Hill (CA)

(21) Appl. No.: 16/277,281

(22) Filed: Feb. 15, 2019

Related U.S. Application Data
(60) Provisional application No. 62/747,898, filed on Oct. 19, 2018.

(51) Int. Cl. G01N 33/98; G01N 33/497; A61B 5/08; B01L 3/00

(52) U.S. Cl. CPC G01N 33/98; G01N 33/4972; G01N 2001/2244; B01L 3/5027; A61B 5/082

(57) ABSTRACT

Systems and methods detect a target analyte in a breath sample. The system includes a sensor device and an analyzer. The sensor device includes a breath capture mechanism for capturing the target analyte from the breath sample. The captured analyte is transferred to a testing solution. The sensor device includes a receptor for selectively binding the target analyte. The receptor and the bound analyte together form a receptor-analyte complex. The sensor device also includes a sensor. The sensor includes at least one electrode. A surface of the at least one electrode binds the receptor-analyte complex when the sensor is exposed to the testing solution containing the target analyte. The analyzer analyzes the sensor to determine a target analyte level. The analyzer includes an electronics subsystem for determining a change in an electrical property of the sensor caused by the presence of the receptor-analyte complex. The analyzer generates the target analyte level based on the change in the electrical property.

### TECHNICAL FIELD

The following relates generally to analyte detection, and in particular to systems and methods for detecting a target analyte in a breath sample.

### INTRODUCTION

Approaches directed to the detection of analytes such as drugs and other chemical species in individuals are important to the promotion of safety and health among individuals and populations. This may be particularly true in jurisdictions where drugs such as Cannabis have become legalized. Many substances exist that can cause impairment. Impairment in drivers and dangerous working environments such as construction and mining sites may be particularly concerning due to the potential for injury caused by an impaired individual to oneself or others. Effective testing for the presence of analytes linked to impairment or other physiological conditions may be critical to ensure the health and safety of individuals.

Existing systems and methods for analyte detection may have high costs of manufacture or implementation. Timeliness of detection and provision of results may be a drawback of existing systems and methods. These systems may also lack portability and durability. Such drawbacks may prevent effective adoption of existing analyte detection systems in field operations, applications, and environments, and other point-of-care uses.

Existing analyte detection systems using samples other than breath (“non-breath samples”) such as oral fluid, urine, and blood may be invasive and difficult to sample. Analysis of the non-breath sample may require the use of laboratory equipment.

Existing systems may require tightly controlled conditions for testing, operation, and effective analysis. In some cases, convenient and effective testing may be important in environments where a trade-off between functionality and portability, durability, and ease-of-use is required or desired.

Existing methods of analyte detection, such as for tetrahydrocannabinol (THC), may be limited to testing of blood, oral fluid, or other non-breath samples. In some cases, analyte detection may require laboratory equipment that is neither durable nor portable to point-of-care or field applications. Further, existing analyte detection systems may not provide convenient ways to test for multiple analytes.

Accordingly, there is a need for systems, methods, and devices that overcome at least some of the disadvantages of existing analyte detection techniques.

### SUMMARY

A system for detecting a target analyte in a breath sample is provided herein. The system includes a sensor device and an analyzer. The sensor device includes a breath capture mechanism for capturing the target analyte from the breath sample. The captured analyte is transferred to a testing solution. The sensor device includes a receptor for selectively binding the target analyte. The receptor and the bound analyte together form a receptor-analyte complex. The sensor device includes a sensor. The sensor includes at least one electrode. A surface of the sensor binds the receptor-analyte complex when the sensor is exposed to the testing solution containing the target analyte. The analyzer analyzes the sensor to determine a target analyte level. The analyzer includes an electronics subsystem for determining a change in an electrical property of the sensor caused by the presence of the receptor-analyte complex. The analyzer generates the target analyte level based on the change in the electrical property.

The target analyte may include a non-volatile compound.

The target analyte may include tetrahydrocannabinol.

The electrical property may be impedance, capacitance, current, resistance, or voltage.

The target analyte level may be determined by comparing the electrical property of the sensor with the target analyte present to a reference.

The reference may be generated internally to the system.

The system may include a fluidics system for transporting a fluid component of the sensor device.

The sensor device may include a microfluidic chip.

The at least one electrode may include a working electrode, a reference electrode, and a counter electrode.

The electronics subsystem may determine the change in the electrical property using at least one of a pulsed technique, an impedance spectroscopy technique, and a voltamperometric technique.

The system may include a sensor cartridge. The sensor cartridge may include the sensor and the receptor.

The sensor cartridge may include the breath capture mechanism.

The sensor cartridge may include a microfluidics system for transporting a fluid component of the sensor device.

A method of detecting a target analyte in a breath sample is provided herein. The method includes capturing the target analyte from the breath sample. The method includes transferring the target analyte to a testing solution. The method includes specifically binding the target analyte to form a receptor-analyte complex. The method includes measuring a change in an electrical property caused by the presence of the receptor-analyte complex. The method also includes determining a target analyte level using the change in the electrical property.

The electrical property may be impedance, capacitance, current, resistance, or voltage.

The steps of capturing the target analyte, transferring the target analyte, and selectively binding the target analyte may be carried out on a microfluidic chip.

A method of evaluating a breath sample for a target analyte is provided herein. The method includes capturing the target analyte from the breath sample. The method includes exposing a sensor to a testing solution. The testing solution includes the captured analyte. The sensor includes at least one electrode and a receptor for specifically binding the target analyte. The method also includes specifically binding the target analyte in the testing solution to form a receptor-analyte complex on the surface of the sensor. The presence of the receptor-analyte complex generates a measurable change in an electrical property of the sensor.

The method may include transferring the captured analyte to the testing solution.

The method may include determining a target analyte level for the breath sample using the change in the electrical property of the sensor.

Other aspects and features will become apparent, to those ordinarily skilled in the art, upon review of the following description of some exemplary embodiments.

### BRIEF DESCRIPTION OF THE DRAWINGS

The drawings included herewith are for illustrating various examples of articles, methods, and apparatuses of the present specification. In the drawings:

FIG. 1 is a block diagram of a system for detecting a target analyte in a breath sample, according to an embodiment;

FIG. 2 is a perspective view of a system for detecting a target analyte in a breath sample, in accordance with an embodiment;

FIG. 3 is a graphical representation of an oxidation of a redox pair at a working electrode surface, according to an embodiment;

FIG. 4 is top view of a sensor having a three-electrode setup, according to an embodiment;

FIG. 5 is a top view of an example electrode geometry for use in a sensor, according to an embodiment;

FIG. 6 is a top view of electrode geometries, in accordance with a plurality of embodiments;

FIG. 7 is a side view of a sensor before and after exposure to a breath sample containing a target analyte, according to an embodiment;

FIG. 8 is a graphical representation of a representative differential pulse voltammetry (DPV) input signal, according to an embodiment;

FIG. 9 is a graphical representation of a DPV curve, according to an embodiment;

FIG. 10 is a graphical representation of a representative DPV curve for various concentrations of THC using an impedimetric immunosensor, according to an embodiment;

FIG. 11 is a top view of a schematic diagram of a sensor cartridge having a microfluidic system, according to an embodiment;

FIG. 12 is a side view of a sensor cartridge having a layered design, according to an embodiment; and

FIG. 13 is a flowchart of a method of detecting a target analyte in a breath sample, according to an embodiment.

### DETAILED DESCRIPTION

Various apparatuses or processes will be described below to provide an example of each claimed embodiment. No embodiment described below limits any claimed embodiment and any claimed embodiment may cover processes or apparatuses that differ from those described below. The claimed embodiments are not limited to apparatuses or processes having all of the features of any one apparatus or process described below or to features common to multiple or all of the apparatuses described below.

The following relates generally to analyte detection, and more specifically to systems, methods, and sensor cartridges for detection of analytes in a breath sample. The systems, methods, and sensor cartridges may use impedimetric, capacitive, or voltammetric sensing in the detection of the target analyte.

The systems of the present disclosure may be particularly advantageous. The systems may provide a less invasive alternative to existing analyte detection techniques. In particular, the systems analyze a breath sample. The breath sample analysis may be less invasive than sampling other mediums such as urine, oral fluid, or blood. The breath sample may be easier to sample than other non-breath media. Analyte detection performed by the systems of the present disclosure may not require the use of laboratory equipment. The systems may be provide increased portability, durability, and cost-effectiveness of components.

The present disclosure provides a target analyte detection system. The target analyte detection system may be used to detect impairment or recent use. The system may detect impairment of or recent use by a subject by determining a target analyte level for a breath sample provided by the subject. In a particular case, the system determines a concentration of THC in the breath sample. The target analyte concentration may be correlated with or used to determine actual or potential impairment in the subject providing the breath sample. The system may use an impedimetric sensor for analyzing breath samples. In some cases, the analyte detection system may be used in detecting disease, for example by detecting a disease state or disease marker.

The present disclosure provides a sensor cartridge for use in a target analyte detection system. The sensor cartridge may use a microfluidic cartridge design. The microfluidic design may provide for capture, elution, and analysis of the sample in a single chip.

The systems and methods provided herein may use voltammetry techniques to analyze a sample and obtain information about the presence of a target analyte. The systems and methods described herein may use a voltammetric sensor to analyze the impedance of an electrical cell (in the form of a sensor) and determine a target analyte level. The system 100 may use differential pulse voltammetry or electrochemical impedance spectroscopy.

The target analyte detection system measures a change in an electrical property of an electrochemical system. The electrochemical system is implemented using a sensor. The electrical property may be capacitance or impedance. Generally, the electrical property is affected by the presence of the target analyte in the breath sample. The system uses the measured electrical property to determine the target analyte level for the breath sample.

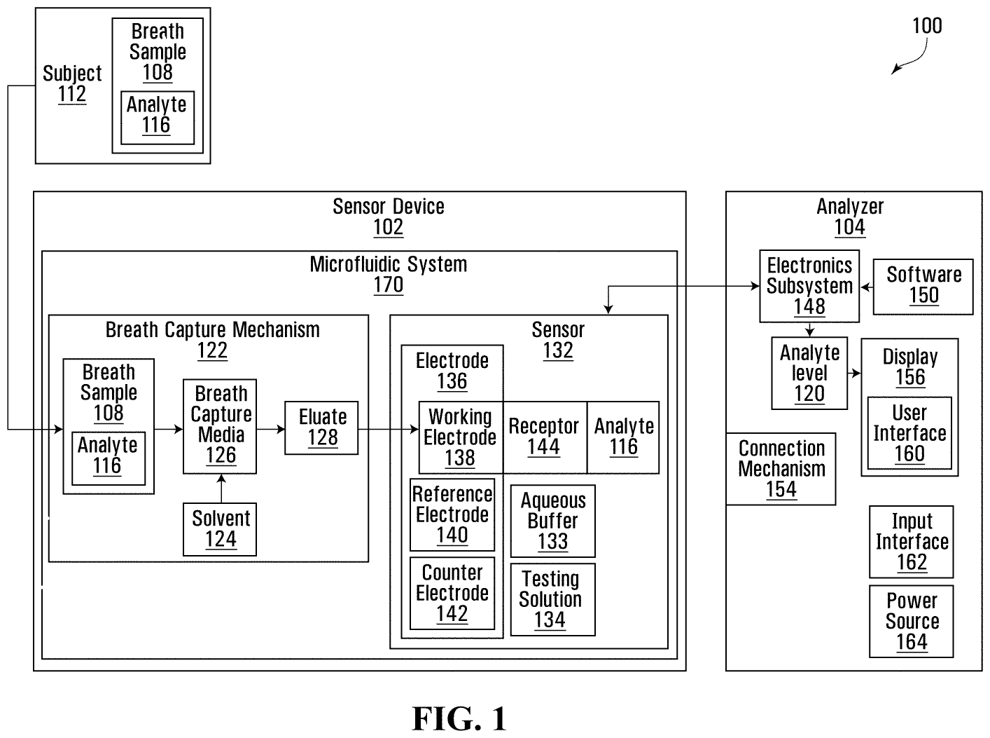

Referring to FIG. 1, shown therein is a system 100 for detecting a target analyte in a breath sample, according to an embodiment. The system 100 may be used to detect impairment of a subject by detecting the presence of a target analyte in a sample provided by the subject. In a particular case, the system 100 may be used to analyze the concentration of THC in a breath sample. The system 100 may be used to determine a concentration of analyte. The concentration of analyte may be correlated with or used to determine recent use of a substance. The concentration of analyte may be correlated with or used to determine actual or potential impairment.

In another case, the system 100 may be used to detect a disease state or disease marker. For example, the system 100 may be used to detect an analyte that is correlated with a particular disease state. The detection of the analyte provides some insight into the presence or absence of the disease state in the subject providing the sample. In some cases, the analyte may be considered a disease marker the presence of which provides some insight into the presence or absence of a particular disease state in the subject.

The system 100 includes an electrochemical system. The electrochemical system is implemented using a sensor (see sensor 132 below). The electrochemical system may include an electrochemical cell. The system 100 analyzes the electrochemical system (i.e. the sensor) after exposure to the breath sample to detect the presence of a target analyte (e.g. analyte 116 described below). The system 100 can compare the analysis of the electrochemical system in the presence of the target analyte to an analysis of the electrochemical system (or another identical or substantially identical electrochemical system) in the absence of the target analyte to detect the target analyte in the breath sample.

The system 100 measures an electrical property of the electrochemical system. The electrical property may be current, voltage, impedance, capacitance, resistance, or the like. The electrical property measurement can be used to determine a target analyte level 120. The system 100 (or a sensor of the system) measuring a particular electrical property may be referred to herein as having a particular format associated with that electrical property. For example, where the system 100 measures impedance, the system 100 (or a sensor thereof) can be said to be operating in an “impedance format”.

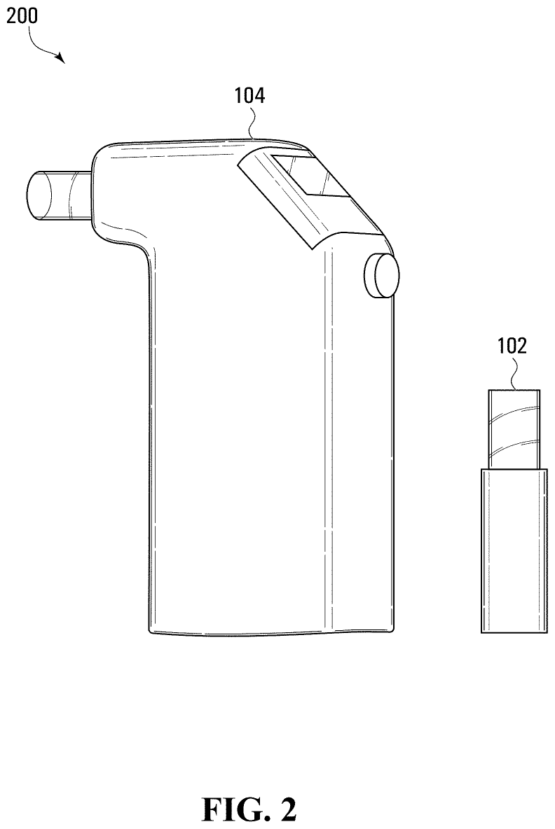

The system 100 includes a sensor device 102 and an analyzer 104. The sensor device 102 may be a sensor cartridge (e.g. as shown in FIG. 2). The sensor device 102 does not have to be one unit. For example, components and functionalities of the sensor device 102 may be implemented in one or more physically separate modules. The modules may be cartridge-type devices (“cartridges”). The sensor device 102 may include one or more reagent cartridges containing one or more reagents.

In an embodiment, the sensor device 102 may be housed in a first device and the analyzer 104 in a second device (e.g. as shown in FIG. 2). Such a two-device embodiment of the system 100 may simplify use. The first device housing the sensor device 102 may be a sensor cartridge, as described above. In another embodiment, the sensor device 102 and the analyzer 104 may be embodied in a single device (e.g. housed in a single device).

In further embodiments of system 100, various components and functionalities of the sensor device 104 and the analyzer 104 may be combined, spread across, embodied, or implemented in any number of physical devices or apparatus.

The physical devices of system 100 (i.e. when implemented as a plurality of physical devices) may communicate via wireless or wired communication. For example, the physical devices may communicate via Bluetooth. In a particular example, the system 100 may include a sensor device and a smart phone (or similar device) implementing an application. The sensor device may communicate with the smart phone application via Bluetooth or other wireless communication.

The sensor device 102 receives a breath sample 108 from a subject 112. Upon receiving the breath sample 108 from the subject 112, the sensor device 102 is exposed to analyte present in the breath sample 108.

A graphical rendering 200 of the system 100 is shown in FIG. 2, according to an embodiment. The rendering 200 shows the sensor device 102 and the analyzer 104. The sensor device 102 as shown is a sensor cartridge. The rendering 200 shows a portable, hand-held embodiment of the system 100. Other embodiments of system 100 may not be hand-held. The embodiment of the system 100 shown in rendering 200 may be particularly advantageous in applications where portability of the system 100 is desirable or required.

Referring again to FIG. 1, once the sensor device 102 is exposed to the breath sample 108, the sensor device 102 is provided to the analyzer 104. Providing the sensor device 102 to the analyzer 104 facilitates analysis of the sensor device 102 by the analyzer 104. Providing the sensor device 102 to the analyzer 104 may include inserting the sensor device 102 or a portion thereof into the analyzer 104 (e.g. a sensor cartridge). The sensor device 102 may be inserted into the analyzer 104 prior to or after collecting the breath sample 108. A portion of the sensor device 102 (e.g. a portion to be analyzed) may be exposed to one or more components of the analyzer 104 to facilitate the analysis.

The analyzer 104 analyzes the sensor device 102 to determine a target analyte level 120 for the breath sample 108. The target analyte level 120 may be correlated to an impairment level or recent use level of the subject 112. The correlation may be performed by an operator of the system 104, such as by reference to a standard, or may be performed by the analyzer 104 as part of the analysis.

The sensor device 102 may be disposable. The sensor device 102 may be a one-use cartridge. The one-use cartridge can be used to collect and provide a signal to for a single breath sample.

The sensor device 102 includes a housing for housing various components of the system 100 (such as sensor 132 and breath capture mechanism 122 described below). For example, the sensor device 102 may conveniently house breath capture and sensing functionalities in a single housing. In another embodiment, the breath capture and sensing functionalities may be housed in separate physical devices (e.g. separate cartridges). The sensor device 102 may be designed to facilitate fluid transport between components for effective analysis by the system 100.

The system 100 includes a breath sample 108. The breath sample 108 is provided by a subject 112. Generally, the subject 112 is an individual for which an operator of the system 100 wants to determine or measure the presence or absence of a particular physiological condition. The physiological condition may be impairment, an elevated or reduced level of a target analyte present in the subject 112, or simply the presence of a particular substance at any level in the subject 112. The subject 112 may be an individual suspected of impairment, such as a worker/employee or a vehicle operator. The subject 112 may be an individual that may be subjected to routine testing, such as an employee working in a dangerous environment (e.g. mining, construction). The subject 112 may be a patient with an existing medical condition (e.g. diabetes) that can be monitored using the system 100.

The breath sample 108 includes a target analyte 116. It should be noted that while the breath sample 108 may be referred to throughout the present disclosure as containing the target analyte 116, it is to be understood that a given breath sample from a subject may not include the target analyte 116 (as the target analyte is not present in that particular breath sample).

The target analyte 116 is a substance or chemical constituent that is detected by the system 100. The target analyte 116 may be linked to or correlated with a physical or physiological condition or state in the subject 112, such that it is of practical value (e.g. public safety, health) to detect the presence or level of the target analyte 116 in the subject 112.

The target analyte 116 may be a non-volatile compound. The target analyte 116 may be a drug or a chemical constituent or component of a drug. The target analyte 116 may be a psychoactive component of a drug responsible for or known to cause impairment or altered cognition. The target analyte 116 may be a metabolite.

In a particular embodiment, the target analyte 116 may be tetrahydrocannabinol (THC). In another embodiment, the target analyte 116 may be glucose. In another embodiment, the target analyte 116 may be methamphetamine. In another embodiment, the target analyte 116 may be alcohol. In another embodiment, the target analyte 116 may be cocaine. In another embodiment, the target analyte 116 may be a vitamin or metabolites of the vitamin. The vitamin may be vitamin D.

In some cases, the system 100 may be configured to detect a plurality of analytes 116. For example, the system 100 may implement a multi-analyte panel. The multi-analyte panel may be a drug panel. The multi-analyte panel may be a disease panel. The disease panel may provide for testing or detection of a plurality of diseases by the system 100.

The system 100 includes a target analyte level 120. The target analyte level 120 is determined by the analyzer 104. The target analyte level 120 is determined from the presence of the target analyte 116 in the breath sample 108. The target analyte level 120 may be a concentration of the target analyte 116. The system 100 may be configured to determine the target analyte level 120 for a plurality of analytes 116.

The system 100 may measure or determine the target analyte level 120 in a variety of ways.

In an embodiment, an electrical property of the electrochemical system of system 100 is measured (e.g. capacitance, impedance, etc.) with the target analyte 116 present. The measured electrical property is compared against a reference. The reference may be a known standard or reference curve, a reference or look-up table, or the like, constructed independently from the current electrochemical system. To generate the reference, various known analyte levels can be run on a representative sensor. The representative sensor may be almost identical to the sensor used to analyze the breath sample 108.

In another embodiment, an electrical property of the electrochemical system is measured with the target analyte 116 present. At the same time, known analyte levels 120 of analyte 116 are run on one or more reference sensors in the system. There may be a number of reference sensors (e.g. 3-5 sensors). The reference sensors are used to construct an internal reference. This may include use of the analyzer 104 to generate the internal reference (e.g. an internal reference curve). The reference may be a standard curve, a reference or look-up table, or the like. The response from the sensor 132 is compared against the reference sensors. This approach to measuring the target analyte level 120 may correct for environmental factors such as humidity, temperature, or other environmental stressors.

The target analyte level 120 may be used in a secondary determination. The secondary determination may be performed by the analyzer 104 or by a human operator. The secondary determination may correspond to the presence or absence of a physical or physiological condition of interest in the subject 112. Generally, the secondary determination may be based on a quantitative standard for the condition of interest. The secondary determination may be an impairment level. The secondary determination may be a recent use level. In some cases, the system 100 may be designed to determine the presence or absence of impairment or the level of impairment as part of the analysis of the breath sample 108.

As described above, the breath sample 108 containing the target analyte 116 is provided to the sensor device 102. The sensor device 102 includes a breath capture mechanism 122. The breath capture mechanism 122 is positioned in the sensor device 102 such that the breath capture mechanism 122 (or components thereof) is exposed to the breath sample 108 upon provision to the sensor device 102 by the subject 112.

The breath capture mechanism 122 is configured to capture the target analyte 116 from the breath sample 108 when the breath capture mechanism 122 is exposed to the breath sample 108. The breath capture mechanism 122 may be configured to transfer the captured analyte 116 to a solvent 124.

The breath capture mechanism 122 (or components thereof) may be contained in a separate cartridge. In an embodiment, the breath capture mechanism 122 (or components thereof) may be contained or implemented in a mask.

The breath capture mechanism 122 includes a breath capture media 126. The breath capture media 126 is configured to capture the target analyte 116 from the breath sample 108. The breath capture media 126 may be contained in a separate cartridge. In another embodiment, the breath capture media 126 (or components thereof) may be contained or implemented in a mask.

The breath capture media 126 may be a filter. The breath capture media 126 may be a hydrophobic filter. The breath capture media 126 may include a PVDF membrane. The PVDF membrane may be hydrophobic or hydrophilic. The breath capture media 126 may include a PTFE membrane. The PTFE membrane may be hydrophobic. The breath capture media 126 may include C18 catch media. The breath capture media 126 may include silica beads. The breath capture media 126 may include electrostatic filters. The breath capture media 126 may include a hydrophobic polycarbonate membrane. The breath capture media 126 may include a nitrocellulose membrane. The breath capture media 126 may include a filter membrane. The filter membrane may include a woven filtration media.

The breath capture media 126 may have a pore size of 0.22 um. In a particular embodiment, the breath capture media 126 may include a hydrophobic PVDF membrane and a 0.22 um pore size. In another embodiment, the breath capture media 126 may include a hydrophilic PVDF membrane and a 0.22 um pore size. In another embodiment, the breath capture media 126 may include a hydrophobic PTFE membrane and a 0.22 um pore size.

The sensor device 102 includes the solvent 124 (also known as an “extraction solution”). The solvent 124 is applied to the breath capture media 126 to wash the captured analyte 116 off the breath capture media 126. The solvent 124 extracts the captured analyte 116 from the breath capture media 126. The breath capture mechanism 122 may perform the washing via an elution mechanism to generate an eluate 128 containing the solvent 124 and the target analyte 116.

The solvent 124 may be a hydrophobic solvent. The solvent 124 may be propanol. The solvent 124 may be ethanol. The solvent 124 may be methanol. The solvent 124 may be isopropanol. The solvent 124 may be DMSO. The solvent 124 may be DMF. The process of washing the breath capture media 126 with the solvent 124 generates the eluate 128 containing the solvent 124 and the target analyte 116.

The solvent 124 may be selected based on the polarity of the solvent 124 and the polarity of the target analyte 116.

For example, glucose is polar and therefore has a high solubility in polar solvents (1200 g/L in water). Therefore, a polar solvent (e.g. water) may be used as the solvent 124 where the target analyte 116 is glucose (or another polar analyte).

THC, cocaine, and vitamin D have very low solubility in polar solvents (2.8 g/L, 1.8 g/L, and <1 g/L, respectively). Therefore, a non-polar solvent may be used as the solvent 124 where the target analyte 116 is THC, cocaine, or Vitamin D (or another analyte having a low solubility in polar solvent).

The solvent 124 may be selected experimentally. In some cases, the solvent 124 may end up in the testing solution. In such cases, the solvent 124 may be selected based on a favourable interaction with the receptor 144 (e.g. antibodies, aptamers, etc.). A solvent 124 having an unfavourable interaction with the receptor 144 may be left out of or removed from the testing solution in order to promote effective detection conditions.

In an embodiment, the breath capture mechanism 122 may include a breath condensing apparatus. The breath condensing apparatus condenses the breath sample 108. The breath condensing apparatus may be used instead of the breath capture media 126. The breath condensing apparatus may eliminate the need for a washing step (and solvent 124) performed by the breath capture mechanism 122. The breath condensing apparatus may simplify the breath capture process performed by the system 100.

In another embodiment, the breath capture mechanism 122 may include directly bubbling the breath sample 108 through the solvent 124. Bubbling may be used instead of first capturing the breath sample 108 on a filter.

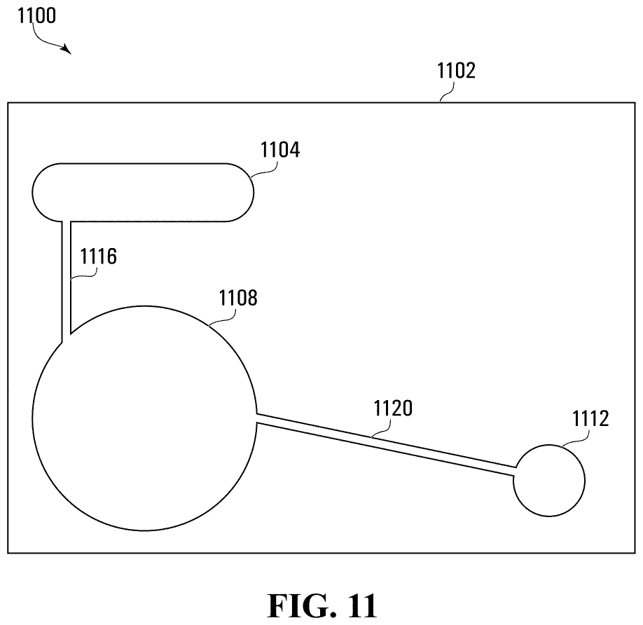

The sensor device 102 includes a sensor 132. The sensor 132 is positioned in the sensor device 102 such that the sensor 132 is exposed to the breath sample 108. The breath sample 108 may be transported to the sensor 132 from the breath capture mechanism 122 via a microfluidic system (e.g. MFS 1100 of FIG. 11).

The sensor 132 may be positioned in an area of the sensor device 102 that is connected to the breath capture mechanism 122 via a channel (for transporting a fluid). In other embodiments, the breath sample 108 may be transported from the breath capture mechanism 122 to the sensor 132 manually by an operator or technician. In another embodiment, the sensor 132 may be positioned in the sensor device 102 such that the breath sample 108 does not need to be transported from the breath capture mechanism 122 in order to be analyzed.

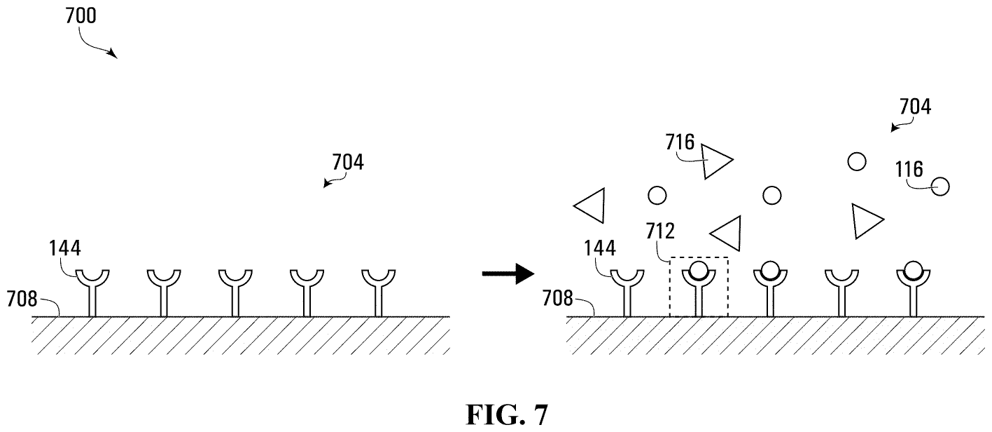

The sensor 132 may operate in a liquid environment. The sensor 132 includes an aqueous buffer 133 (e.g. aqueous buffer 704 of FIG. 7). The aqueous buffer 133 provides an environment that promotes binding of the receptor 144 to the target analyte 116. The aqueous buffer 133 may maintain the pH of the testing solution within a certain range, even if external chemicals or conditions may theoretically push the pH outside of this range. The aqueous buffer 133 solution may be phosphate buffered saline (PBS). The aqueous buffer 133 may be tris buffered saline (TBS).

The system 100 includes a testing solution 134. The testing solution 134 includes the target analyte 116. The testing solution 134 is the final solution in which the sensor 132 is tested. The composition of the testing solution 134 may depend the configuration and format of the system 100 and/or the type or format of the sensor 132. The testing solution 134 may be a liquid or a gas. In an embodiment, the testing solution 134 may include the aqueous buffer 133.

In an impedance format, the testing solution 134 may include a redox probe (e.g. Fe(CN)₆⁻³ and Fe(CN)₆⁻⁴). The testing solution 134 may include a supporting electrolyte for boosting system conductivity.

In a capacitance format, the testing solution 134 may be simple deionized water. In some capacitance format embodiments, a testing solution may not be required and thus not included.

The testing solution 134 may include contents from earlier steps performed by the system 100. For example, the testing solution 134 may include the extraction solution 124 used to extract the target analyte 116 from the breath capture media 126. The extraction solution 124 may be an organic solvent (e.g. methanol) that does not contribute to the analysis. In some cases, the target analyte 116 may be removed from the extraction solution 124 and transferred to the testing solution 134 prior to being analyzed.

In an embodiment, the sensor 132 is an impedimetric sensor and the receptor 144 is an antibody. The antibody is not sensitive to the breath extraction solvent 124. The target analyte 116 is extracted off the breath capture media 126 and the eluate 128 is transferred directly to the sensor 132 for analysis. In some cases, the eluate 128 may be transferred to the testing solution 134 prior to exposing the sensor 132 to the testing solution 134. The testing solution 134 includes some proportion of the solvent 124, which may be a non-polar solvent (e.g. methanol), and some proportion of aqueous buffer 133 (e.g. PBS). If the system 100 is using a faradaic setup, the testing solution 134 may also include a redox probe (e.g. K₃Fe(CN)₆ and K₄Fe(CN)₆).

In another embodiment, the sensor 132 is an impedimetric sensor and the receptor 144 is an antibody. The antibody is sensitive to the breath extraction solvent 124. The target analyte 116 is extracted off the breath capture media 126 and transferred from the extraction solvent 124 to the testing solution 134. The testing solution 134 is provided to the sensor 132 for analysis. In such a case, the testing solution 134 includes the aqueous buffer 133 (PBS). If the system 100 is using a faradaic setup, the testing solution 134 also includes the redox probe (e.g. K₃Fe(CN)₆ and K₄Fe(CN)₆).

In another example, the sensor 132 is a capacitive sensor and the receptor 144 is an antibody. The antibody is not affected by the breath extraction solvent 124. The antibody may function in a liquid environment. The target analyte 116 is extracted off the breath capture media 126 and the eluate 128 is transferred to the sensor 132 for analysis. The testing solution 134 includes some proportion of the solvent 124, which may be a non-polar solvent (e.g. methanol), and some proportion of the buffer 133 (e.g. PBS).

In another example, the sensor 132 is a capacitive sensor and the receptor 144 is an antibody. The antibody is affected by the breath extraction solvent 124. The antibody runs in a liquid environment. The target analyte 116 is extracted off the breath capture media 126. The target analyte 116 is transferred from the extraction solvent 124 to the testing solution 134. The testing solution 134 (containing the target analyte 116) is transferred to the sensor 132 for analysis. The testing solution 134 includes the aqueous buffer 133 (e.g. PBS).

In yet another example, the sensor 132 is a capacitive sensor and the receptor 144 is an antibody. The antibody is affected by the breath extraction solvent 124. The antibody does not need to run in a liquid environment. The target analyte 116 is extracted off the breath capture media 126. The target analyte 116 is transferred from the extraction solvent 124 to the testing solution 134. The testing solution 134 is provided to the sensor 132 for incubation. The target analyte 116 is allowed to incubate (i.e. bind) with the antibodies. The testing solution 134 is removed for analysis. In such a case the testing solution 134 is composed of air.

The sensor 132 may be a voltammetric sensor. The sensor 132 may be an impedimetric sensor. The sensor 132 may be a capacitive sensor. The sensor 132 may be an immunosensor.

Upon exposure to the breath sample 108, the sensor 132 generates a signal that can be analyzed by the analyzer 104. The sensor 132 may include an electrochemical cell. The electrochemical cell may be a voltammetric cell. The electrochemical cell can be analyzed by the system 100 (i.e. the analyzer 104) to provide information on the presence of the target analyte 116 in the breath sample 108.

The sensor 132 includes an electrode 136. The electrode 136 may be a conductor through which electricity enters or leaves the sensor 132.

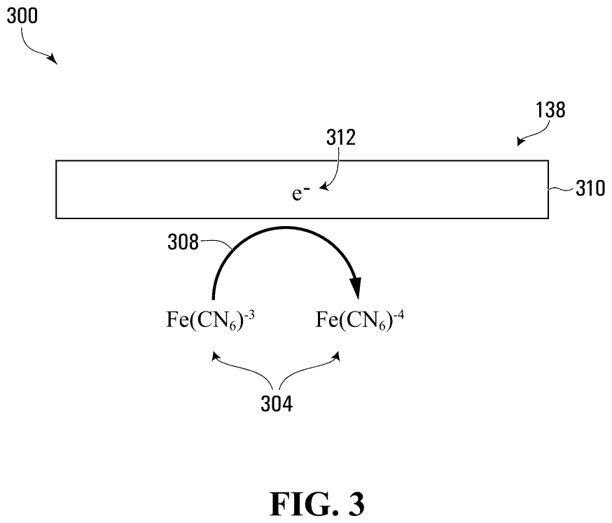

The electrode 136 may provide a surface (e.g. electrode surface 308 of FIG. 3) that serves as a location where oxidation-reduction equilibrium can be established between the electrode 136 and the testing solution 134. In variations, the electrode 136 may include a single electrode or a plurality of electrodes. The plurality of electrodes may include one or more quality control (QC) electrodes. The plurality of electrodes may include sensing multiple electrodes.

In an embodiment where the sensor 132 is an impedimetric sensor, the electrode 136 provides a source of electrons for the oxidation and reduction of the redox species. The testing solution 134 contains a redox probe and the aqueous buffer 133.

In an embodiment where the sensor 132 is a capacitive sensor, the electrode 136 includes a plurality of electrodes. The electrodes serve as “parallel plates” for a dielectric layer to create a capacitor. The testing solution 134 may be the aqueous buffer 133 (e.g. PBS) or simply air.

The electrode 136 may include various electrode materials. The electrode 136 may include gold. The electrode 136 may include silver. The electrode 136 may include silver chloride (AgCl). The electrode 136 may include platinum. The electrode 136 may include nickel. The electrode 136 may include chromium. The electrode 136 may include titanium. The electrode 136 may include aluminum. The electrode 136 may include carbon. The electrode 136 may include doped silicone. The electrode 136 may include one or more conductive polymers. The conductive polymer may be polyaniline. The conductive polymer may be polypyrrole. The electrode 136 may include a combination of electrode materials.

The electrode 136 may be manufactured using a screen-printing technique to generate a screen-printed electrode (SPE). The electrode 136 may be manufactured using thermal vapor deposition. The electrode 136 may be manufactured using e-beam deposition.

In an embodiment, the electrode 136 may include a layer of chromium covered by a layer of gold. The chromium and gold layers may each be 50 nm thick. In other embodiments, the electrode 136 may include pure gold, platinum, carbon, or conductive polymer. The electrode 136 may be manufactured using lithography to pattern a glass slide, followed by thermal evaporation to deposit the gold onto the surface in a desired pattern.

The electrode 136 may be configured in a variety of multi-electrode setups such as a two-electrode, three-electrode, or four-electrode setup. The size and shape of the electrodes in the multi-electrode setup may vary.

The electrode 136 may include a working electrode 138. In some cases, the electrode 136 may include a plurality of working electrodes 138. The working electrode 138 is an electrode in the electrochemical cell on which the reaction of interest is occurring. The working electrode 138 may be in contact with the testing solution 134.

The working electrode 138 applies a desired potential or current in a controlled manner. The working electrode 138 may facilitate a transfer of charge to and from the testing solution 134 (e.g. in an impedimetric format).

Referring now to FIG. 3, shown therein is a reaction 300 occurring at the working electrode 138, according to an embodiment. The reaction 300 can be measured by the analyzer 104 to determine the target analyte level 120. A redox species (or redox pair) 304 in the testing solution 134 is oxidized/reduced 308 at a surface 310 of the working electrode 138. The redox species may be a ferrocyanide/ferricyanide redox pair. The redox species 304 is oxidized/reduced 308 as electrons 312 become available due to current flow. Changes in the surface 310 of the working electrode 138 may make electrons 312 more or less available. Availability of the electrons 312 may make the oxidation/reduction 308 of the redox species 304 easier or more difficult. The change in ease of reduction/oxidation 308 of the redox species 304 can be measured by the analyzer 104.

Referring again to FIG. 1, the working electrode 138 may be made of an inert material. The inert material may include gold, silver, platinum, nickel, chromium, titanium, and/or carbon. The size and shape of the working electrode 138 may vary.

The electrode 136 may include a reference electrode 140. In some cases, the electrode 136 may include a plurality of reference electrodes 140. The reference electrode 140 may be a half cell with a known reduction potential. The reference electrode 140 acts as a reference in measuring and controlling the potential of the working electrode 138. The reference electrode 140 may not pass any current. The reference electrode 140 may have a stable and well-known electrode potential.

The reference electrode 140 may be used as a point of reference in the electrochemical cell for the potential control and measurement. The high stability of the reference electrode potential may be reached by employing a redox system with constant (buffered or saturated) concentrations of each participants of the redox reaction. Moreover, the current flow through the reference electrode 140 may be kept close to zero (ideally, zero). Keeping the current flow through the reference electrode 140 may be achieved by using a counter electrode (e.g. counter electrode 142 described below) to close the current circuit in the electrochemical cell together with a very high input impedance on an electrometer (e.g. >100 GOhm).

The electrode 136 may include a counter electrode 142. In some cases, the electrode 136 may include a plurality of counter electrodes 142. The counter electrode 142 passes current to balance the current observed at the working electrode 138. The counter electrode 140 may be used to close the current circuit in the electrochemical cell. The counter electrode 142 may be made of an inert material. The inert material may include platinum, gold, graphite, or carbon. The counter electrode 142 may not participate in the electrochemical reaction.

In an embodiment, each of the working electrode 138, the reference electrode 140, and the counter electrode 142 is exposed to the testing solution 134. For example, the testing solution 134 may cover the working electrode 138, the reference electrode 140, and the counter electrode 142.

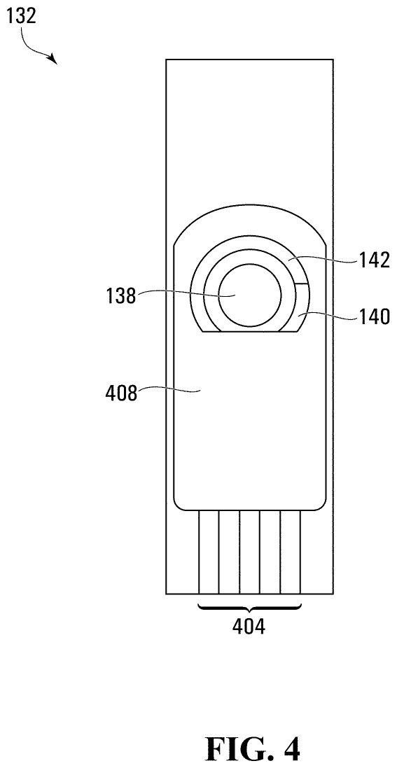

Referring now to FIG. 4, shown therein is an embodiment of the sensor 132 having a three-electrode system.

The sensor 132 includes the working electrode 138, the reference electrode 140, and the counter electrode 142.

The sensor 132 includes a cover piece 408. The cover piece 408 may be composed of plastic. The cover piece 408 covers the leads of the electrodes 138, 140, 142, preventing damage to the leads (e.g. scratching). The cover piece 408 may also provide cosmetic or aesthetic appeal to the sensor 132. In variations of the sensor 132, the cover piece 408 may be omitted.

The sensor 132 includes a connector 404 to connect to the analyzer 104 (the electronics subsystem 148 described below).

In an embodiment, the reference and counter electrodes 140, 142 may be implemented as a single second electrode (i.e. a two-electrode system along with the working electrode 138). The second electrode may supply electrons and provide a reference potential. The second electrode acts as the other half of the electrochemical cell (with the working electrode 138). The second electrode has a known potential with which to gauge the potential of the working electrode 138. The second electrode balances the charge added or removed by the working electrode 138.

In another embodiment, the electrode 136 is implemented as a four-electrode setup. In the four-electrode setup a working sense lead may be decoupled from the working electrode (e.g. working electrode 138).

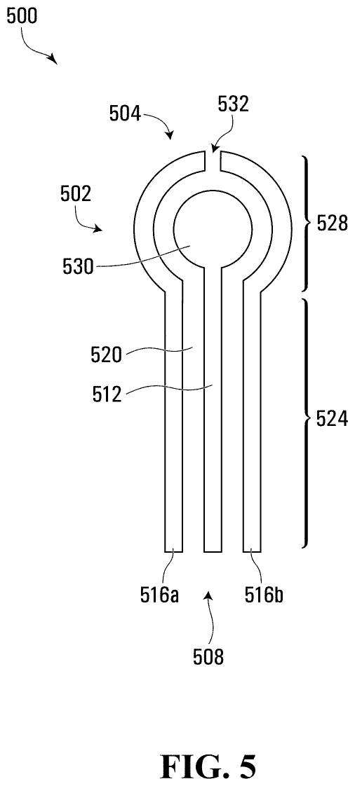

The electrode 136 may have a variety of electrode geometries. Referring now to FIG. 5, shown therein is an electrode geometry 500 for an electrode 502, according to an embodiment. The electrode 502 may be used as electrode 136 of FIG. 1. The electrode 502 has a hairpin-type shape.

The electrode 502 includes a first end 504 and a second end 508.

The electrode 502 includes an inner segment 512 and an outer segment 516. The inner segment 512 and outer segment 516 may run approximately parallel to one another with a space 520 in between the inner and outer segments 512, 516.

The inner segment 512 and outer segment 516 each include a narrow segment 524 and a wide segment 528. The narrow segment 524 is located at the first end 504. The narrow segment 524 may be elongated and straight.

The wide segment 528 is located at the second end 508. The wide segment 528 is rounded. In other embodiments, the wide segment 528 may be rectangular. The wide segment 528 of the inner segment 512 is a solid circle 530 of electrode material. In embodiments, where the wide segment 528 is rectangular, the wide segment 528 of the inner segment 512 may be a solid rectangle of electrode material.

The outer segment 516 includes a first outer segment 516a and a second outer segment 516b separated by a gap 532. In other embodiments, the outer segment 516 may be a single continuous segment. The outer segment 516 may be a thin strip (i.e. thinner) relative to the inner segment 512.

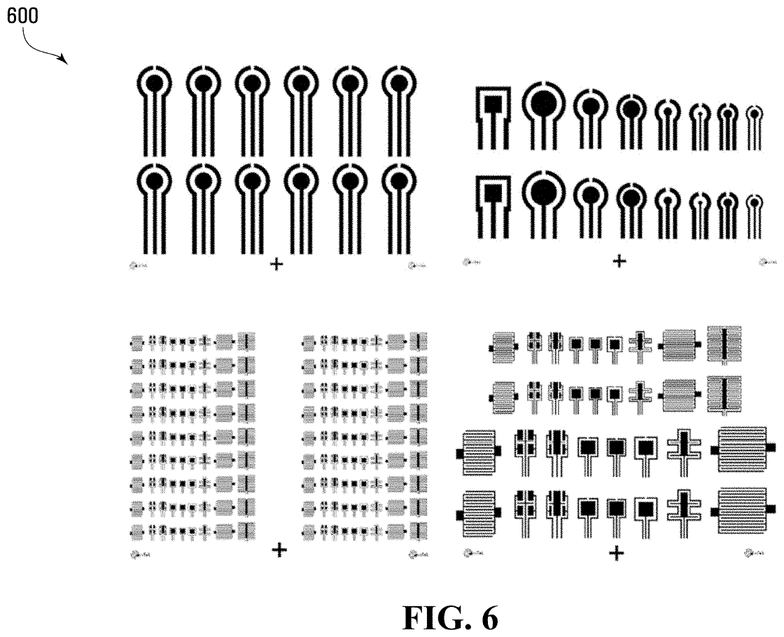

Referring now to FIG. 6, shown therein are various possible electrode geometries for the electrode 136, according to embodiments.

Referring again to FIG. 1, the sensor 132 includes a receptor 144. The receptor 144 is a target analyte-binding molecule. The receptor 144 is configured to selectively bind the target analyte 116.

The receptor 144 binds the analyte 116 to form a receptor-analyte complex (e.g. receptor-analyte complex 712 of FIG. 7). The receptor-analyte complex binds to a surface of the sensor 132. As used herein, the term “binding of the receptor-analyte complex to a surface of the sensor” and variants thereof refers to the state of the receptor-analyte complex being bound or immobilized relative to the surface of the sensor 132. In other words, the term refers to the state of the complex being immobilized relative to the surface regardless of whether the analyte is binding to a receptor that is already bound (i.e. affixed) to the surface, the receptor is binding to an analyte that is already bound (i.e. affixed) to the surface, an unbound receptor binds to an unbound analyte which then binds the surface as a complex, an unbound receptor binds to an unbound analyte which then binds to another receptor bound (i.e. affixed) on the surface as a complex, or other such variation on the order of binding, affixed components, intermediates, etc.

As used herein, “a surface of the sensor” or variants thereof when used in reference to a site for binding the receptor-analyte complex refers to any surface of the sensor 132 suitable for binding the receptor-analyte complex in such a way that may cause a measurable change in an electrical property that can be used to determine the analyte level 120.

In an embodiment (e.g. an impedance format), the surface of the sensor 132 that binds the receptor-analyte complex may be an electrode surface (e.g. a surface of electrode 136).

In another embodiment (e.g. a capacitance format), the surface of the sensor 132 that binds the receptor-analyte complex may be a gap between electrodes. The surface may be a substrate that the electrodes are made on, such as glass or silicon. The surface may be a material deposited between the electrodes (e.g. polymer, silicon dioxide).

Referring now to FIG. 7, shown therein is a representation of a binding event 700 resulting from exposure of the sensor 132 to the breath sample 108, according to an embodiment. The sensor 132 includes an aqueous buffer 704. The aqueous buffer 704 provides an environment suitable for promoting the binding of the receptor 144 to the target analyte 116.

The receptor 144 is configured to selectively bind the target analyte 116. The receptor 144 does not bind a non-analyte molecule 716. The receptor 144 may be selected or engineered to selectively bind the target analyte 116. The receptor 144 having a bound analyte 116 forms a target analyte-receptor complex 712 (or “receptor-analyte complex”).

The receptor 144 may include a plurality of homogeneous receptors for binding a single analyte 116. The receptor 144 may include a plurality of heterogeneous receptors. The heterogeneous receptors may include a first receptor subset configured to bind a first analyte and a second receptor subset configured to bind a second analyte. Having a heterogenous receptor arrangement may advantageously allow the sensor device 102 to be analyzed for multiple analytes 116.

The system 100 may use a variety of techniques or assay formats for binding the receptor 144 to the target analyte 116. For example, the receptor 144 may bind the target analyte 116 using a direct assay, indirect assay, capture (“sandwich”, “immunometric”) assay, or competitive assay technique.

For the direct assay, the target analyte 116 is immobilized on a surface of the sensor 132. The receptor 144 is introduced. The receptor 144 is a labelled anti-analyte antibody (or other receptor). A magnitude of response from the label is determined. The label may be fluorescence, phosphorescence, chemical reaction, or the like. The magnitude of response is used to determine the target analyte level 120.

The indirect assay may include a two-step binding process using a primary receptor and a secondary receptor. The secondary receptor is a labelled receptor. The primary receptor (receptor 144) is specific for the analyte 116. The secondary receptor is specific for the primary receptor. The secondary receptor may be a polyclonal anti-species antibody. The target analyte 116 is immobilized on a surface of the sensor 132. The target analyte 116 may be immobilized using target analyte-coated wells. The primary receptor is introduced. This may include incubating the primary receptor with the target analyte-coated wells. The primary receptor binds the target analyte 116. Next, the labelled secondary receptor is introduced. The secondary specifically binds to the primary receptor. A magnitude of response from the labelled secondary antibody can be determined. The label may be fluorescence, phosphorescence, chemical reaction, or the like. The magnitude of response is used to determine the target analyte level 120.

The sandwich (or immunometric) assay uses two receptors specific to the target analyte 116 to capture or “sandwich” the target analyte 116 for detection utilizing different epitopes on the target analyte 116 to form the sandwich. The sandwich assay may exhibit a direct correlation between target analyte concentration and substrate response. In an embodiment, a capture antibody (receptor 144) is immobilized on the surface of the sensor 132 (e.g. a surface of the electrode 136). The capture antibody may be coated on the surface of the sensor 132. The capture antibody is specific to the target analyte 116. The target analyte 116 is introduced and binds the capture antibody (a first incubation). During a second incubation, the target analyte 116 is bound by a detection receptor. The detection receptor is also specific to the target analyte 116. The detection receptor may be bound by a labelled secondary receptor specific to the detection receptor. The label may be fluorescence, phosphorescence, chemical reaction, or the like. The magnitude of labelled secondary receptor response can be determined. The magnitude of labelled receptor response is used to determine the target analyte level 120 (i.e. the concentration of captured target analyte molecules).

For the competitive assay, the target analyte 116 in a sample competes for limited receptor binding sites with target analyte 116 conjugated to a reporter receptor. This produces an inverse relationship between target analyte 116 concentration and substrate turnover. Competitive assays may use a single receptor to a low molecular weight target analyte, generally less than 10,000 Daltons. During incubation, samples with high target analyte content may result in unlabeled target analyte being bound in greater amounts than conjugated target analyte. When substrate against the label is added to the assay to develop signal, samples with a high target analyte 116 concentration generate a lower signal than samples containing low target analyte 116 concentration, yielding an inverse correlation between target analyte 116 concentration in the sample and signal in the assay. This relationship can then be used to extrapolate target analyte 116 concentration in an unknown sample from a standard curve. In particular, the competitive assay format may be effective for a low molecular weight target analyte 116 with a limited number of epitopes or receptor-binding sites, such as the small molecule THC.

The receptor 144 may be a biomolecule. The receptor 144 may be an antibody. The antibody may be a monoclonal antibody. The antibody may be a polyclonal antibody. The antibody may be a recombinant antibody. The antibody may be/include immunoglobulin G (IgG). In some cases, the receptor 144 may include only an antigen-binding fragment (“Fab”) of the antibody (e.g. IgG Fab). The receptor 144 may be a synthetic antibody.

The receptor 144 may be an aptamer. The aptamer may be a nucleic acid aptamer (e.g. DNA aptamer). The aptamer may be a peptide aptamer. The aptamer may function similarly to an antibody.

The receptor 144 may be a polymer. The polymer may be a specialized polymer. The specialized polymer may be a molecular imprinted polymer. The molecular imprinted polymer may include an antigen imprinted into polymer.

The receptor 144 or analyte 116 (or other receptor), as the case may be, may be affixed or immobilized to a surface of the sensor 132. The receptor 144 or analyte 116 may be immobilized to the surface using passive adsorption, direct linking, or the like.

The receptor 144 may be affixed or immobilized to a surface 708 of the electrode 136 (e.g. in an impedance format). The surface 708 may be a surface of the working electrode 138. The receptor 144 may be immobilized to the surface of the working electrode 138 via a linker.

The receptor 144 may be linked to the surface 708 of the electrode 136 via passive adsorption (passively adsorbing). The receptor 144 may be linked to the surface 708 of the electrode 136 via direct linking.

In an embodiment, the receptor 144 may be an anti-THC antibody attached to the surface 708 of a gold working electrode 138. The receptor 144 may be immobilized to the surface 708 of the gold working electrode 138 by using DTT (dithiothreitol) to convert the sulfide bonds within the antibody receptor 144 into thiol groups that naturally bond with gold surfaces. The receptor 144 may be immobilized to the surface 708 of the gold working electrode 138 by creating a BSA (bovine serum albumin) layer on the gold surface 708 of the working electrode 138 and immobilizing the antibodies 144 to the BSA layer via EDC/NHS (1-Ethyl-3-(3-dimethylaminopropyl)carbodiimide/N-Hydroxysuccinimide) coupling. The receptor 144 may be immobilized to the surface 708 of the gold working electrode 138 by using a DSP (dithiobis(succinimidyl propionate)) cross linker to attach the antibody receptor 144 to the gold surface 708 of the working electrode 138.

The receptor 144 may be positioned in a gap in between electrodes (e.g. in a capacitance format). The receptor 144 may be bound to the gap. The receptor 144 may be bound to a substrate that the electrodes are on. The receptor 144 may be bound to a material deposited in the gap, such as polymer or silicon dioxide.

In some embodiments, the receptor 144 may be immobilized around the electrode 136.

The sensor 132 may function by measuring impedimetric changes of the working electrode 138 in a liquid medium. When the target analyte 116 is introduced near to a surface of the sensor 132 including the receptor 144 (e.g. surface 708), the target analyte 116 binds selectively with the receptor 144. Binding of the target analyte 116 to the receptor 144 generates the target analyte-receptor complex 712 on the surface 708 of the working electrode 138.

The sensor 132 may be a faradaic sensor or a non-faradaic sensor. The faradaic and non-faradaic sensors may use EIS.

In an embodiment, the sensor 132 is a faradaic sensor. The faradaic sensor approach uses a redox probe in solution (i.e. the testing solution 134). The limitation of the oxidation and reduction of the redox species at the working electrode surface is measured.

In an embodiment, the sensor 132 is a non-faradaic sensor. The non-faradaic sensor approach does not use a redox probe. Instead, the addition of the target analyte 116 results in the displacement of testing solution molecules from the immediate region, changes detectable through associated change in capacitance and, consequently, the impedance.

Presence of the target analyte-receptor complex 712 causes an increase in impedance of the working electrode 138. The increase in impedance can be correlated to the number of bound analyte molecules 116 and thus the concentration of the target analyte 116 in solution. Thus, by measuring the current running through the electrode 136, the system 100 can determine the concentration of the target analyte 116 in the breath sample 108.

The sensor 132 may function in a gas medium (e.g. in a capacitance format). In such an embodiment, the receptor 144 may still bind the target analyte 116 in a liquid medium. The capacitance of the sensor 132 is measured in the gas medium. The gas medium may be plain air.

Various components shown or described as part of the sensor device 102 of FIG. 1 may be implemented (e.g. housed) in one or more physically separate modules (e.g. cartridges). For example, the breath capture mechanism 122 may be housed in a first module and the sensor 132 housed in a second module. In another variation, subcomponents of the breath capture mechanism 122 may be separated into multiple modules. For example, the solvent 124 and the breath capture media 126 (e.g. a filter) may be separated into multiple modules. Similarly, subcomponents of the sensor 132 may be separated into multiple modules.

Referring again to FIG. 1, the analyzer 104 includes an electronics subsystem 148. The electronics subsystem 148 may be configured to implement a voltammetry technique. The voltammetry technique includes measuring the current as the potential is varied to obtain information about the presence of the target analyte 116 in the breath sample 108. The voltammetric method may provide qualitative and quantitative information regarding the target analyte 116 with high precisions (<1%), sensitivity and wide linear dynamic range.

The electronics subsystem 148 includes electronic hardware for controlling an electrochemical cell (e.g., the sensor 132) and performing electroanalytic sensing. The electronics subsystem 148 includes an electric circuit. In an embodiment, the electronics subsystem 148 may maintain a potential of the working electrode 138 at a constant level with respect to the reference electrode 140 by adjusting a current at the counter electrode 142.

The electronics subsystem 148 may apply a time-dependent or frequency-dependent potential to the working electrode 138, changing the potential of the working electrode 138 relative to a fixed potential of the reference electrode 142. The resulting current, flowing between the working electrode 138 and the counter electrode 140, may be measured as a function of the potential. The electronics subsystem 148 may measure electrical current between the working electrode 138 and the counter electrode 140 induced by a redox reaction.

If the redox species is oxidized at the working electrode 138, resulting electrons pass through the electronics subsystem 148 to the counter electrode 140. On the contrary, if the redox species is reduced at the working electrode 138, the current flows from the counter electrode 140 to the working electrode 138.

The electronics subsystem 148 may obtain information about the target analyte 116 by measuring the current as the potential is varied. The electronics subsystem 148 may obtain information about the target analyte 116 by measuring the potential as the current is varied. The electronics subsystem 148 may apply a specific voltage profile to the working electrode 138 as a function of time and the current produced by the sensor 132 is measured. The electronics subsystem 148 may include a potentiostat configured to perform some of the operations. The potentiostat may apply variable potentials to the working electrode 138 relative to the reference electrode 142 while measuring the current that flows as a result of the electrode 136 reaction. Depending on the method, the electronics subsystem 148 may apply a reducing potential and/or an oxidation potential.

The electronics subsystem 148 applies a voltage signal to the sensor 132. The electronics subsystem 148 measures a resulting current in the sensor 132. The electronics subsystem 148 may determine an impedance value of the sensor 132, which is directly related to the current. The electronics subsystem 148 may maintain a desired voltage at the working electrode 138 with reference to the reference electrode 142 by imputing current into the counter electrode 140 at any given time.

The pattern of the voltage signal applied by the electronics subsystem 148 may vary. The electronics subsystem 148 may be configured to apply a voltage signal according to a differential pulse voltammetry (DPV), electrochemical impedance spectroscopy (EIS), or cyclical voltammetry (CV) technique. In an embodiment, the electronics subsystem 148 may be configured to apply a voltage signal including a series of increasing pulses (e.g. a DPV technique).

The electronics subsystem 148 may analyze the sensor 132 using a pulsed technique. The pulsed technique may include Differential Pulse Voltammetry (what we use currently), Square Wave Voltammetry, Normal Pulse Voltammetry, Reverse Normal Pulse Voltammetry, Differential Normal Pulse Voltammetry, or Differential Pulse Amperometry.

The electronics subsystem 148 may analyze the sensor 132 using an impedance spectroscopy technique. The impedance spectroscopy technique may include Galvano Electrochemical Impedance Spectroscopy, Potentio Electrochemical Impedance Spectroscopy, Staircase Galvano Electrochemical Impedance Spectroscopy, Staircase Potentio Electrochemical Impedance Spectroscopy, or Potentio Electrochemical Impedance Spectroscopy Wait.

The electronics subsystem 148 may analyze the sensor 132 using a voltamperometric technique. The voltamperometric technique may include Cyclic Voltammetry, Linear Sweep Voltammetry, Chronoamperometry/Chronocoulometry, Chronopotentiometry, Staircase Voltammetry, Large Amplitude Sinusoidal Voltammetry, or AC Voltammetry.

Using EIS, the electronics subsystem 148 may measure the resistance and capacitance properties of the sensor 132 via application of a sinusoidal AC excitation signal. The excitation signal may be in a range of approximately 2-10 mV. The electronics subsystem 148 may implement EIS by applying a sinusoidal signal of varying frequencies to the electrode 136 in the testing solution 134 and measuring a resulting current. The testing solution 134 includes the buffer 133 (e.g. PBS). If the sensor 132 is a faradaic sensor, the testing solution 134 also includes the redox probe. If the sensor 132 is a non-faradaic sensor, the testing solution 134 does not include the redox probe. The testing solution 134 may also include a supporting electrolyte for improving conductivity of the sensor 132 (sensor conductivity).

The electronics subsystem 148 may generate an impedance spectrum. The impedance spectrum is obtained by varying frequency over a defined range. The electronics subsystem 148 may calculate a capacitance and resistance of the sensor 132 by measuring in-phase and out-of-phase current responses. The electronics subsystem 148 may use EIS to detect a binding event such as the target analyte 116 binding the receptor 144 on the surface of the electrode 136 (e.g. surface 708 of FIG. 7). The electronics subsystem 148 may use EIS to determine a base resistivity of the sensor 132.

The electronics subsystem 148 may calculate a corresponding impedance for the electrode 136. The electronics subsystem 148 may plot an imaginary component versus a real component. By doing so, the electronics subsystem 148 can generate a Nyquist plot.

The Nyquist plot includes an arc section and a linear section. By measuring the diameter of the arc, the electronics subsystem 148 can determine a charge transfer resistance or resistivity of the electrochemical system.

The EIS technique may be based on measuring the frequency-dependent impedance of the sensor 132 followed by analysis of the impedance. The electronics subsystem 148 may use continuous wave impedance spectroscopy. Continuous wave impedance spectroscopy may measure the impedance in the frequency domain by applying a single sinusoidal voltage of small amplitude with a defined frequency to the sensor 132 and recording the corresponding current. Applying a discrete set of different frequencies may provide a frequency spectrum of the impedance of the sensor 132. The electronics subsystem 148 may use Fourier transform impedance spectroscopy. Fourier Transform impedance spectroscopy may use a transient excitation signal applied to the sensor 132. The response of the sensor 132 can be monitored in the time domain and subsequently transformed to the frequency domain by Fourier Transformation providing the frequency dependent impedance of the sensor 132.

The electronics subsystem 148 may analyze the sensor 132 using cyclical voltammetry (CV). Using CV, the potential of the working electrode 138 may be ramped linearly versus time. When the potential reaches a set potential, the potential ramp of the working electrode 138 may be inverted. The inversion may occur multiple times. Scanning the potential in both directions may provide an opportunity to explore the electrochemical activity of species generated at the electrode 136. This may provide an advantage of over other voltammetric techniques.

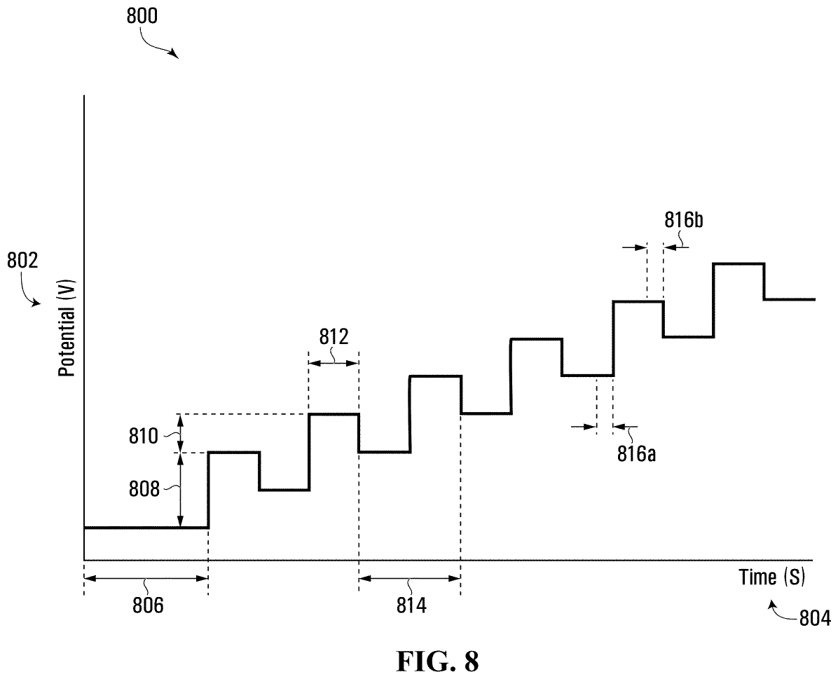

Referring now to FIG. 8, shown therein is a graphical representation of an example applied voltage input signal 800, according to an embodiment. The signal 800 represents the application of a DPV technique that can be used in system 100. The signal 800 includes a series of increasing pulses. The signal 800 is represented on a plot having a Y-axis 802 corresponding to a potential measured in volts and an x-axis 804 corresponding to time measured in seconds. The signal 800 includes a quiet time 806. The signal includes a pulse amplitude 808. The signal 800 includes a step size E 810. The signal 800 includes a pulse width 812. The signal 800 includes a pulse period 814. The signal 800 includes a sample period 116 (sample periods 116a, 116b). The foregoing measurements are DPV input settings.

The electronics subsystem 148 may be configured to control a number of DPV parameters including start voltage, stop voltage, step size 810, pulse height 808, pulse period 814, and pulse width 812. In a particular embodiment, the electronics subsystem 148 applies a voltage signal to the sensor 132 according to a DPV technique including a start voltage of approximately 300 mV, a stop voltage of 300 mV, a step size 810 of 4 mV, a pulse height 808 of 10 mV, a pulse period 814 of 200 ms, and a pulse width 812 of 50 ms.

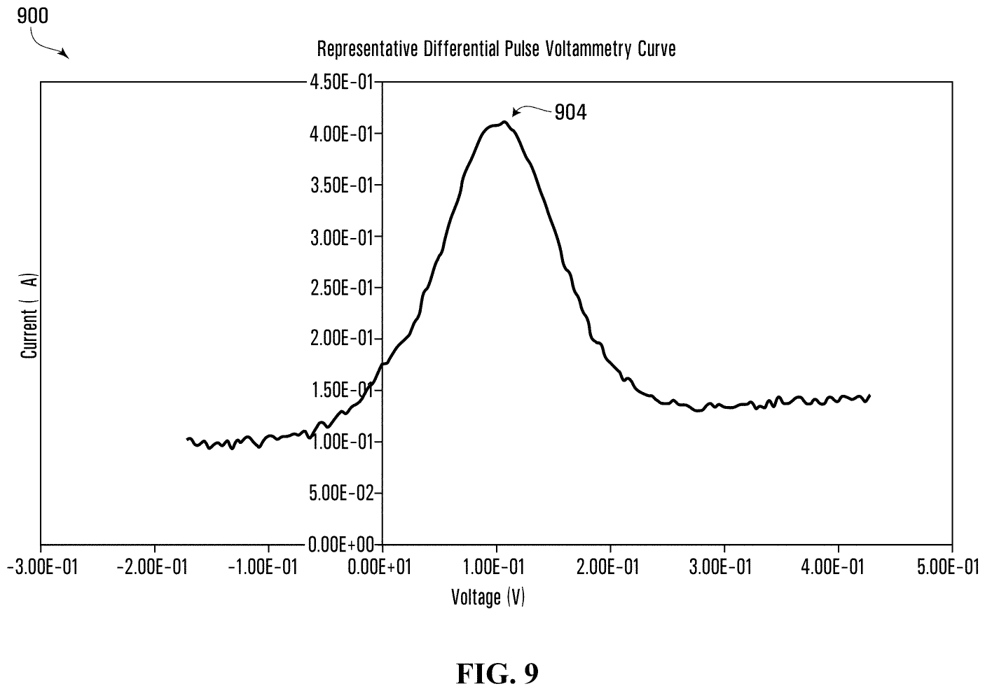

The applied voltage signal generates a current response in the electrode 136. The current response may take the form of a redox peak 904 as shown in graph 900 of FIG. 9. The redox peak 904 is the result of redox species (e.g. redox species 304 of FIG. 3) in the testing solution 134 being oxidized and reduced at the surface (e.g. surface 310 of FIG. 3) of the working electrode 138 as electrons (e.g. electrons 312 of FIG. 3) become available due to current flow. The testing solution 134 is a combination of the buffer 133 to maintain the pH within a range that is acceptable for the receptor 144, the redox probe for generating a signal, and may include a supporting electrolyte for increasing conductivity of the testing solution 134.

Changes in the surface of the working electrode 138, such as formation of analyte-receptor complex (e.g. analyte-receptor complex 712 of FIG. 7) can make the electrons more or less available and thus make the oxidation and reduction of the redox species easier or more difficult. If reduction/oxidation becomes easier, the DPV/redox peak 904 increases in magnitude. If reduction/oxidation becomes more difficult, the DPV/redox peak 904 decreases in magnitude. Accordingly, the DPV/redox peak 904 magnitude is inversely proportional to the concentration of the target analyte 116 in solution.

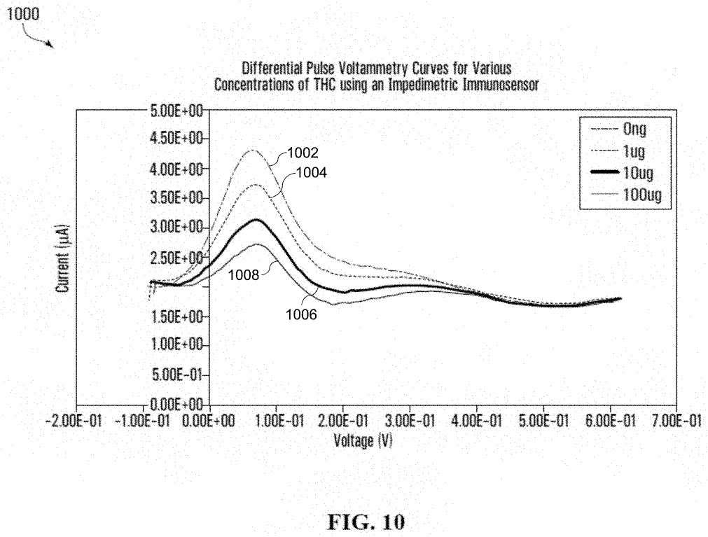

Referring now to FIG. 10, shown therein is a DPV plot 1000, according to an embodiment. The plot 1000 includes a representative series of DPV peaks for various concentrations of THC (i.e. analyte 116) is shown in FIG. 10.

The plot 1000 shows a response of a sensor 132 when exposed to various concentrations of THC. Curve 1002 illustrates a sensor response when 0 ng/mL of THC is present. Curve 1004 illustrates a sensor response when 1 ug/mL of THC is present. Curve 1006 illustrates a sensor response when 10 ug/mL of THC is present. Curve 1008 illustrates a sensor response when 100 ug/mL of THC is present.

The electronic subsystem 148 may implement one or more of the above control and measuring functions and techniques using a potentiostat. The potentiostat may be a miniaturized potentiostat. The miniaturized potentiostat may advantageously allow the analyzer 104 to be smaller and potentially more portable. The potentiostat analyzes or measures the change in impedance of the sensor 132. The potentiostat may apply a certain voltage signal to the sensor 132 and measure a resulting current. The potentiostat may apply a certain current signal to the sensor 132 and measure a resulting potential. The resulting current directly relates to the impedance of the electrochemical cell. The potentiostat includes an electric circuit for controlling the potential across the cell by sensing changes in its resistance, varying accordingly the current supplied to the system: a higher resistance will result in a decreased current, while a lower resistance will result in an increased current, in order to keep the voltage constant. The potentiostat may maintain a desired voltage at the working electrode with reference to the reference electrode by imputing current into the counter electrode at any given time. The potentiostat may be configured to interface with a computing device and operate through a dedicated software package.

The system 100 may use post-processing of data (measuring the area under the curve instead of the height of the peak). The post-processing of data may be performed by the electronics subsystem 148.

In an embodiment, the electronics subsystem 148 may measure the area under the curve instead of the peak. For example, the electronics subsystem 148 may perform/calculate an integral of the area under each curve to determine the target analyte/THC levels. This would use a capacitive charge-discharge technique. The electronics subsystem 148 in such an embodiment is not measuring the change in impedance. The electronics subsystem 148 is measuring a change in charge of a capacitor.

One or more aspects, components, or functions of the electronics subsystem 148 (e.g. the potentiostat) may be controlled by software 152.

The electronics subsystem 148 may include or be communicatively linked to a computing device including a processor and a memory. The computing device may facilitate analysis, measurement, translation, storage, or conversion of data related to the detection of the target analyte 116 in the breath sample 108.

The analyzer 104 includes a connection mechanism 154. The connection mechanism 154 may be part of or linked to the electronics subsystem 148. The connection mechanism 154 may promote secure coupling or mating of the sensor device 102 (or some component thereof) and the analyzer 104 to facilitate analysis by the electronics subsystem 148. The connection mechanism 152 may be configured to mate or couple with a connection mechanism of the sensor device 102. In some cases, the connection mechanism 152 may promote proper alignment of components in the sensor device 102 with components of the analyzer 104 for effective analysis of the sensor device 102.

The analyzer 104 may include a display 156. The display 156 may provide a user interface 160. The user interface 160 may provide or display results of the analysis of the sensor device 102. The display 156 may allow an operator to manage or control one or more steps of the process. The display 156 may be an LED screen.

The analyzer 104 may include an input interface 162. The input interface 162 may include one or more buttons. The input interface 162 may include a touchscreen interface included as part of the display 156. The input interface 162 may allow an operator of the analyzer 104 to provide information to the analyzer 104 or control certain operations of functions of the analyzer 104.

In some embodiments, the display 156 and/or the input interface 162 may be contained in a separate device. The device may be a laptop, smartphone, android device, etc.

The analyzer 104 includes a power source 164. The power source 164 provides power to the electronics subsystem 148 and other components of the analyzer 104. The power source 164 may be a battery. In some embodiments, the power source 164 may provide power to one or more components of the sensor device 102 (e.g. power components in a cartridge).

The system 100 may include an optical particle counter. The optical particle counter may be included within the electronics subsystem 148 of the analyzer 104. The optical particle counter may characterize the number of exhaled breath particles in the breath sample 108 of the subject 112. Since the target analyte 116 is carried in the breath particles, the optical particle counter output may be used to normalize the data with respect to the number of particles produced by various subjects 112, which can be highly variable.

In an embodiment, the system 100 is adapted to detect a second non-volatile molecule present in the exhaled breath particles (breath sample 108) for normalization.

In an embodiment, the system 100 may be implemented using a semi-conductor-type design. The system 100 may include the receptor 144 immobilized onto a gate region of a transistor. The target analyte 116 binding the receptor 144 (e.g. THC binding the antibody) acts like an applied gate voltage, changing the conductivity of the source-drain channel, resulting in a change in current. The change in current can be measured and correlated to a target analyte level (e.g. THC concentration).

The sensor device 102 includes a microfluidic system 170. The microfluidic system 170 transports and holds fluid at multiple stages of the analysis. The microfluidic system 170 exposes the breath sample 108 to the solvent 124. The microfluidic system 170 transports the solvent 124 (including the target analyte 116) to the sensor 132. In variations, the sensor device 102 may use a macrofluidics system for transporting one or more fluid components of the sensor device 102.

The microfluidic system 170 may include a plurality of reservoirs for holding fluid at various stages of operation. The reservoirs may be various sizes. The reservoirs may be selectively sized for a particular purpose or function in the operation of the system 100.

In an embodiment, the sensor 132 may be positioned in the sensor device 102 such that the solvent 124 does not need to be transported (e.g. the sensor 132 is positioned below the breath capture media 126). For example, the breath capture mechanism 122 may function to condense the breath sample 108. By condensing the breath sample 122, the need for liquid transport (e.g. by the microfluidic system 170) may be removed.

Referring now to FIG. 11, shown therein is a microfluidic system (MFS) 700, according to an embodiment. The MFS 1100 may be the microfluidic system 170 of FIG. 1. The MFS 1100 includes a substrate 1102. The substrate 1102 provides a supporting material on or in which a system of reservoirs and channels can be formed or fabricated.

The MFS 1100 includes a solvent reservoir 1104. The solvent reservoir 1104 contains a solvent for extracting a target analyte (e.g. analyte 116 of FIG. 1) from the breath capture filter (e.g. breath capture media 126 of FIG. 1).

The MFS 1100 includes a filter reservoir 1108. The filter reservoir 1108 contains the breath capture filter. The filter reservoir 1108 may be open both on the top and bottom to allow a breath sample (e.g. breath sample 108 of FIG. 1) to flow into and out of the filter.

The MFS 1100 includes a sensor reservoir 1112. The third reservoir 1112 contains a sensor (e.g. sensor 132 of FIG. 1). The sensor may be an impedimetric sensor or a capacitive sensor.

The MFS 1100 includes a first channel 1116. The first channel 1116 connects the solvent reservoir 1104 to the filter reservoir 1108. The first channel 1116 transports the solvent from the solvent reservoir 1104 to the filter reservoir 1108 where the solvent is used to remove the target analyte from the breath capture media/filter.

The MFS 1100 includes a second channel 1120. The second channel 1120 connects the filter reservoir 1108 to the sensor reservoir 1112. The second channel 1120 transports the eluate from the filter reservoir 1108 to the sensor reservoir 1112. Transporting the eluate to the sensor reservoir 1112 facilitates exposure of the sample to the impedimetric sensor.

The MFS 1100 includes a transport mechanism. The transport mechanism facilitates transport of fluid (i.e. the solvent) from the solvent reservoir 1104 to the filter reservoir 1108 via the first channel 1116, and from the filter reservoir 1108 to the sensor reservoir 1112 via the second channel 1120.

The transport mechanism may generate or apply an electric field for transporting the solvent. The electric field attracts the solvent along the channels 1116, 1120. In an embodiment, the transport mechanism includes a plurality of metal pads. The metal pads may be applied above and below the first and second channels 1116, 1120. The transport mechanism applies a voltage to the metal pads, generating the electric field.

In particular, the solvent may be transported from the solvent reservoir 1104 to the breath capture reservoir 1108 by applying voltage to the metal pads above and below the first channel 1116 to create an electric field that attracts the solvent along the channel 1116. The solvent (now containing the target analyte from the filter) may be transported from the breath capture reservoir 1108 to the sensor reservoir 1112 by applying voltage to the metal pads above and below the second channel 1120 to create an electric field that attracts the solvent including the target analyte along the channel 1120.

In another embodiment, the microfluidic system 170 may include one or more peristaltic pumps for pushing and/or pulling various solutions (e.g. the solvent) through the microfluidic chip.

The transport mechanism may be an external peristaltic pump. The use of an external peristaltic pump as the transport mechanism may allow for storage of system reagents (e.g. extraction solvent) outside of the microfluidic chip 1200. The microfluidic system 170 may include an injection mechanism for injecting the reagent into the microfluidic chip 1200. The injected reagent can be transported throughout the chip 1200/MFS 170 using the external peristaltic pump.

The transport mechanism may include a plurality of membrane valves. The membrane valves can be arranged in series to create an “on-chip” peristaltic pump. In a particular case, three or more membrane valves may be placed in series. The membrane valves may be composed of PDMS. The membrane valves may be composed of a phase-changing polymer.

The membrane valves may be actuated externally by applying pressure. The membrane valves may be actuated by applying heat (e.g. for phase-changing polymer membrane valves). The heat may be applied using an internally built resistive heater.

The membrane valves may be cycled open/closed to pump fluid from left to right. For example, the membrane valves may by cycled as follows, where “O” represents an open membrane valve and “C” represents a closed membrane valve:

### OCC->OOC->COO->CCO->CCC

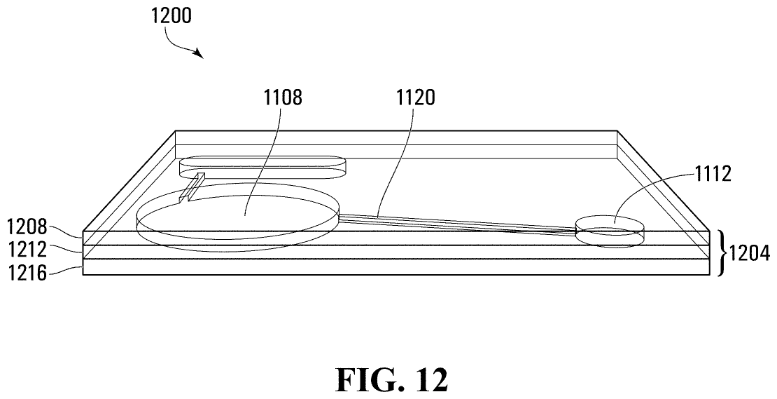

Referring now to FIG. 12, shown therein is a microfluidic chip sensor 1200, according to an embodiment. The microfluidic chip sensor 1200 may be included in a sensor cartridge (e.g. sensor cartridge 102 of FIG. 2). The microfluidic chip sensor 1200 includes a breath capture mechanism (e.g. breath capture mechanism 122 of FIG. 1), a sensor (e.g. sensor 132 of FIG. 1), and a microfluidic system (e.g. microfluidic system 1100 of FIG. 11). The design of the microfluidic chip sensor 1200 may facilitate transfer of a breath sample into aqueous medium. The sensor operates in a liquid environment. The microfluidic chip 1200 promotes capture and transfer of analyte in the breath sample to a solvent. Fluid may be introduced to (e.g. injected) or removed from the microfluidic chip 1200. This may be done via tubing, syringe adapters, or holes in the chip using an external active system (pressure controller, push-syringe or peristaltic pump) or passive approach (e.g. hydrostatic pressure).

The microfluidic chip 1200 includes a plurality of substrate layers 1204. The substrate layers 1204 may be composed of acrylic. The substrate layers 1204 may be composed of glass, silicon, or polymer (e.g. PolyDimethylSiloxane or PDMS). Polymers may provide good bio-chemical performance and low-cost. Polymer may provide transparency of components, elasticity (which may be tuned using cross-linking agents), and reduced cost.

Components of the microfluidic chip 1200 may be connected to outside components by inputs or outputs pierced through the chip 1200. The microfluidic chip 1200 may be formed using a variety of techniques such as deposition and electro-deposition, etching, bonding, injection molding, embossing and soft lithography.

The microfluidic chip 1200 includes three layers 1204 of acrylic substrate. In embodiments, the number and composition of the layers 1204 may vary. The layers 1204 may be sealed together to form the microfluidic chip 1200. In a particular case, the layers 1204 may be heat sealed together by stacking the layers 1204 and placing them into an oven at 350 F for 17 minutes.

The microfluidic chip 1200 may be manufactured using a laser cutting technique. The laser cutting technique may include cutting completely through a material. It may be advantageous for chip 1200 to have at least three layers when using the laser cutting technique.

In an embodiment, the microfluidic chip 1200 includes three layers. A first layer is used to form and contain the channels. A second layer seals the top of the chip 1200. A third layer seals the bottom of the chip 1200. Other variations of the chip 1200 may include two layers or more than three layers. In a two-layer chip embodiment, a first layer includes channels etched into the material. A second layer is placed on top of the first layer and seals the channels.

In a particular embodiment, the chip 1200 may have three layers arranged in a glass-PDMS-glass configuration. The second and third layers (sealing the top and bottom of the chip 1200) are composed of glass. The first layer containing the channels is composed of PDMS.

The microfluidic chip 1200 includes a first substrate layer 1208. The first layer 1208 includes the reservoirs and channels (e.g. reservoirs 1104, 1108, 1112 and channels 1116, 1120 of FIG. 11) of the microfluidic system (e.g. MFS 1100 of FIG. 11). For example, the first layer 1208 may include the solvent reservoir 1104, filter reservoir 1108, and sensor reservoir 1112, the first channel 1116, and the second channel 1120. The reservoirs and channels may be cut or etched into the first layer 1208. The reservoirs and channels may be cut or etched using a laser cutter. The reservoirs and channels may be etched or molded into the first substrate layer 1208.

The first layer 1208 includes the filter. The filter may be held in place by the second and third layers 1212, 1216. The filter is positioned in the filter reservoir 1108.

The microfluidic chip 1200 includes a second layer 1212. The second layer 1212 is positioned below the first layer 1208. The second layer 1212 may be sealed to the first layer 1208. The second layer 1212 includes a plurality of metal pads for transporting the solvent 124 from the solvent reservoir 1104 to the filter reservoir 1108 and from the filter reservoir 1108 to the sensor reservoir 1112. The metal pads may be deposited onto the second substrate layer 1208. The metal pads may be contained within the second substrate layer 1208.

The second layer 1208 also includes the sensor. The sensor may be deposited onto the acrylic substrate of the second layer 1208. The sensor may be contained within the acrylic substrate of the second layer 1208.

The second layer 1208 includes a hole. The hole is located below the filter. In some cases, the second layer 1208 may be a solid piece of substrate/acrylic except for the hole below the filter.

The microfluidic chip 1200 includes a third substrate layer 1216. The third substrate layer 1216 may be composed of acrylic. The third layer 1216 includes a plurality of metal pads for transporting the solvent from the solvent reservoir 1104 to the filter reservoir 1108 and from the filter reservoir 1108 to the sensor reservoir 1112. The metal pads may be deposited onto the third substrate layer 1216. The metal pads may be contained within the third substrate layer 1216.

The third layer 1216 includes a hole. The hole is located above the filter. In some cases, the third substrate layer 1216 may be a solid piece of substrate (e.g. acrylic) except for the hole above the filter.

The chip 1200 may include a valve. The valve opens and closes the hole. In some embodiments, the valve may be contained in the second layer 1212. In other embodiments, the valve is contained in another layer of the chip (e.g. first layer 1208, third layer 1216).

In variations, system components, such as the filter and the sensor, may be positioned differently in the microfluidic chip 1200. Positioning of such components may depend on the number and configuration of layers in the chip 1200, as well as the positioning of other components (e.g. other components that may interact). The valve may be external to the chip 1200. In some cases, the valve may be a plurality of valves. In a particular case, the plurality of valves includes a first valve above the hole and a second valve below the hole.

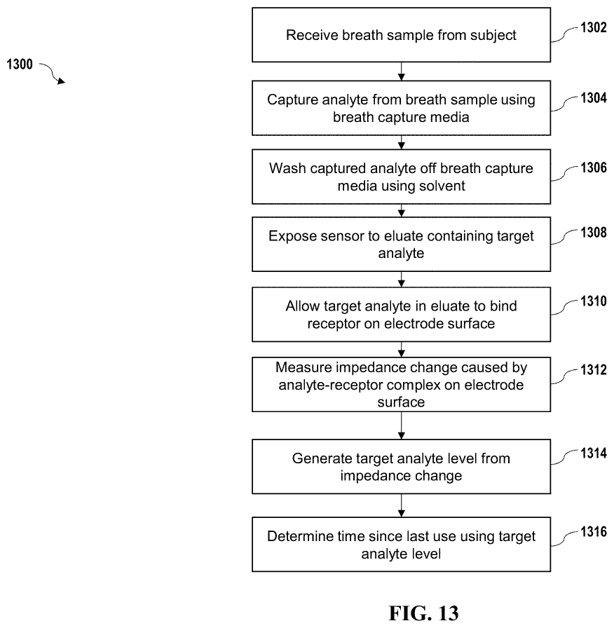

Referring now to FIG. 13, shown therein is a method 1300 of detecting a target analyte in a breath sample, according to an embodiment. The method 1300 may be implemented by system 100. In an embodiment, steps 1302 to 1312 of method 1300 may be performed by the sensor device 102, while steps 1314 to 1318 may be performed by the analyzer 104 (e.g. the electronics subsystem 148).

At 1302, a breath sample is provided by a subject. To provide the breath sample, the subject applies or directs a breath output onto or into the sensor device 102. The breath sample may be provided to a particular part of the sensor device 102 to ensure a usable sample. The breath sample is applied or directed to a breath capture mechanism including a breath capture media (e.g. breath capture mechanism 122, breath capture media 126 of FIG. 1).

At 1304, the breath capture media captures (e.g. filters) the target analyte from the breath sample.

At 1306, the captured analyte is washed off the breath capture media using a solvent. The washing step of 1306 may include or mimic an elution technique. Step 1306 generates an eluate, which is a solution including the solvent and the target analyte.

In some embodiments, steps 1304 and 1306 may be combined into a single step using a breath condensing apparatus. Instead of capturing the breath particles containing the target analyte and washing the target analyte off the breath capture media, the breath sample may be condensed and the condensed breath provided to the sensor.

At 1308, a sensor in the sensor device 102 (e.g. sensor 132) is exposed to the eluate. This may include transporting the eluate from a first location including the breath capture media to a second location including the sensor 132. The transportation of the eluate may be active or passive. By exposing the sensor to the eluate, the target analyte can react with the sensor.

At 1310, the target analyte, having been exposed to the sensor, binds to the sensor. More particularly, the target analyte binds to a receptor on a surface of an electrode of the sensor. Binding of the target analyte to the receptor generates a target analyte-receptor complex on the surface of the electrode.

At 1312, a change in impedance (impedance change) of the sensor can be determined. The impedance change is caused by the presence of the target analyte-receptor complex on the surface of the electrode. The impedance change can be used as input to further analysis steps. The determination of the impedance change involves the provision of the sensor device (having been exposed to the breath sample) to the analyzer 104. In some cases, the sensor device 102 is provided to the analyzer 104 before providing the breath sample. Provision of the sensor device 102 requires that the sensor be analyzable (e.g. readable) by the analyzer 104.

At 1314, the change in impedance can be used to generate a target analyte level for the breath sample. The target analyte level may be a concentration of the target analyte in solution. Generally, the target analyte level can be determined from the impedance change due to the impedance change being proportional to the amount of analyte-receptor complex on the electrode surface.

At 1316, the target analyte level can be used to detect recent use, such as by determining a time since last use. Determination of time since last use may be done manually, such as by a human operator, or automatically by the analyzer.

In another embodiment, the target analyte level from 1314 may be used to determine an impairment level or other secondary determination at 1316. Determination of the impairment level may be done manually, such as by a human operator, or automatically by the analyzer. Determination of the impairment level may include reference to an impairment standard or threshold. The standard or threshold may be similar in function to a blood alcohol level standard for impaired driving. Detection of impairment may be indicated to the operator via the analyzer 104 and may include alerting the operator to a need for subsequent action.

The target analyte detection system 100 may provide various advantages over existing analyte detection systems.

The system 100 and components thereof may have a reduced cost of manufacture and high processability. The components of system 100 may have an increased durability over existing systems which may be particularly advantageous in field applications. Systems used in field applications and environments may be vulnerable to damage. Existing systems may require tightly controlled conditions for operation and effective analysis. This may be because the environment is dangerous, is not tightly controlled, or may be exposed to outside elements. Such environments may include police field applications, construction sites, manufacturing sites, and mining sites. The system 100 may advantageously provide an operator with a portable and durable analyte detection system for use in such environments. The system 100 may advantageously provide a means of non-invasive or minimally-invasive sampling for analyte detection. The system 100 may also provide a reduced-cost and timely approach to analyte detection.

The system 100 may provide an effective system for workplace drug testing. The system 100 may be particularly effective in safety sensitive industries such as mining and construction, by employers looking to be sure employees are not using or under influence at work.

The system 100 may have increased durability over optical methods, which can require carefully calibrated machines that can decrease their durability. Increased durability may be particularly advantageous in point of care or field operations/testing, such as in impairment testing by police (roadside testing) and workplace testing (mining, construction sites). In particular, the analyzer 104 may be portable and durable such that it can be stored in the back of a police cruiser. The system 100, including its components such as the cartridge 102, analyzer 104, and sensor 132, may have an increased ease of manufacturing. The sensor 132 may be manufactured in a cost-effective manner per sensor device 102 (which may include one or more cartridges). The system 100 may provide an improved tradeoff between functionality and cost, durability, and portability compared with existing systems.

Advantageously, the system 100 may be operated by a non-expert, such as a police officer, employer, or patient.

The system 100 may be configured to accept and analyze multiple types of sensor cartridges. For example, a first sensor cartridge may be configured to detect a first analyte, while a second sensor cartridge may be configured to detect a second analyte. Advantageously, the sensor cartridge may be designed to stay substantially the same in structure and function with the exception of the receptor 144 (which is specific to a particular analyte 116). In such cases, the system 100 may advantageously provide a degree of interoperability by having multiple sensor cartridges (directed to different analytes 116) compatible with the analyzer 104. An operator can select an appropriate cartridge 102 for a desired detection operation, acquire the breath sample 108 and provide the cartridge 102 to the analyzer 104. The system 100 may incorporate a plurality of receptor types with each receptor type specific for a particular analyte 116. Using such an arrangement, the system 100 may advantageously provide efficient testing by facilitating detection of multiple analytes 116 using a single cartridge 102. The use of an impedimetric sensor may allow the system 100 to detect a target analyte 116 in a subject without the use of enzyme labels.

The systems and methods described herein refer to the use of electrochemical methods in determining a target analyte level in a breath sample. The use of such methods may include the use of an immunosensor or immunosensing techniques. In variations, other detection techniques using an immunosensor such as colorimetric and fluorescence immunosensing techniques (or any other suitable immunosensing technique) may be used in place of or in addition to electrochemical methods when implementing the systems and methods described herein, which can be modified as required to implement such systems and methods.

While the above description provides examples of one or more apparatus, methods, or systems, it will be appreciated that other apparatus, methods, or systems may be within the scope of the claims as interpreted by one of skill in the art.

1. A system for detecting a target analyte in a breath sample, the system comprising:
a breath capture mechanism for capturing the target analyte from the breath sample, and wherein the captured analyte is transferred to a testing solution;
a sensor device including:
a receptor for selectively binding the target analyte, wherein the receptor and the bound analyte together form a receptor-analyte complex; and
a sensor including:
at least one electrode;
wherein a surface of the sensor binds the receptor-analyte complex when the sensor is exposed to the testing solution containing the target analyte; and
an analyzer for analyzing the sensor to determine a target analyte level, the analyzer comprising an electronics subsystem for determining a change in an electrical property of the sensor caused by the presence of the receptor-analyte complex, and wherein the analyzer generates the target analyte level based on the change in the electrical property.

2. The system of claim 1, wherein the target analyte comprises a non-volatile compound.

3. The system of claim 1, wherein the target analyte comprises tetrahydrocannabinol.

4. The system of claim 1, wherein the electrical property is impedance, capacitance, current, resistance, or voltage.

5. The system of claim 1, wherein the target analyte level is determined by comparing the electrical property of the sensor with the target analyte present to a reference.

6. The system of claim 5, wherein the reference is generated internally to the system.

7. The system of claim 1, further comprising a fluidics system for transporting a fluid component of the sensor device.

8. The system of claim 1, wherein the sensor device comprises a microfluidic chip.

9. The system of claim 1, wherein the receptor is affixed to the surface of the at least one electrode.

10. The system of claim 1, wherein the at least one electrode comprises a working electrode, a reference electrode, and a counter electrode.

11. The system of claim 1, wherein the electronics subsystem determines the change in the electrical property using at least one of a pulsed technique, an impedance spectroscopy technique, and a voltamperometric technique.

12. The system of claim 1, further comprising a sensor cartridge, wherein the sensor cartridge comprises the sensor and the receptor.

13. The system of claim 12, wherein the sensor cartridge further comprises the breath capture mechanism.

14. The system of claim 12, wherein the sensor cartridge further comprises a microfluidics system for transporting a fluid component of the sensor device.

15. A method of detecting a target analyte in a breath sample, the method comprising:
capturing the target analyte from the breath sample;
transferring the target analyte to a testing solution;
selectively binding the target analyte to form a receptor-analyte complex;
measuring a change in an electrical property caused by the presence of the receptor-analyte complex; and
determining a target analyte level using the change in the electrical property.

16. The method of claim 15, wherein the electrical property is impedance, capacitance, current, resistance, or voltage.

17. The method of claim 15, wherein the steps of capturing the target analyte, transferring the target analyte, and selectively binding the target analyte, are carried out on a microfluidic chip.

18. A method of evaluating a breath sample for a target analyte, the method comprising:
capturing the target analyte from the breath sample;
exposing a sensor to a testing solution, wherein the testing solution includes the captured analyte, and wherein the sensor includes at least one electrode and a receptor for selectively binding the target analyte; and
selectively binding the target analyte in the testing solution to form a receptor-analyte complex on a surface of the sensor; and
wherein the presence of the receptor-analyte complex generates a measurable change in an electrical property of the sensor.

19. The method of claim 18, further comprising transferring the captured analyte to the testing solution.

20. The method of claim 18, further comprising determining a target analyte level for the breath sample using the change in the electrical property of the sensor.
